(19) United States
(12) Patent Application Publication
(10) Pub. No.: US 2017/0227779 A1
(43) Pub. Date: Aug. 10, 2017

(54) Head Mounted Display And Wearable Computer

(71) Applicant: KONICA MINOLTA, INC., Chiyoda-ku (JP)

(72) Inventors: Junichi KATO, Shibukawa-shi (JP); Satoru HIROSE, Tondabayashi (JP)

(21) Appl. No.: 15/515,521

(22) PCT Filed: Sep. 11, 2015
(86) PCT No.: PCT/JP2015/075786

§ 371 Date: Mar. 29, 2017

(30) Foreign Application Priority Data
Sep. 30, 2014 (JP) 2014-200953
Sep. 30, 2014 (JP) 2014-201152

(51) Int. Cl. G02B 27/02; G02B 27/01; G06F 3/01; H04N 5/64

(52) U.S. Cl. CPC G02B 27/02; H04N 5/64; G02B 27/017; G02B 27/0179; G06F 3/012; A61M 2205/507

(57) ABSTRACT

A head mounted display with the improved weight balance of an image display unit and an additional device that are supported by a spectacle-type frame is provided. The head mounted display includes: an image display unit including an image display member 2 to be disposed in front of a wearer's eye; a frame including a left front part and a right front part to be disposed on a front side of the wearer's head, a right temple part connected to a right end of the right front part, and a left temple part connected to a left end of the left front part, and supporting the image display unit; a relay unit including a relay circuit board on which a sensor for detecting a state of the wearer's head is mounted, and supported by the right temple part or the left temple part; an external connection cable connected to the relay circuit board; an intermediate cable for connecting between the relay circuit board and the image display unit; and an earphone unit 70 (a second housing 71). Transmission paths for signals/power are formed by the external connection cable, the relay circuit board, and the intermediate cable.

### TECHNICAL FIELD

The present invention relates to a head mounted display (head mounted display) and a wearable computer (wearable computer).

### BACKGROUND ART

In recent years, head mounted displays which are mounted on the wearers' head and enable them to view an image personally, and wearable computers utilizing such displays have been developed. Connecting a head mounted display to a mobile computer forms a computer graphic screen as a virtual image in front of the user's eyes. Thus, a wearable computer whose display can be mounted on the user's head is provided.

Specific examples of the head mounted display include the head mounted display in which an additional device is supported by a spectacle-type frame as disclosed in Patent Literatures 1 and 3 and the head mounted display whose shape is based on the headphone as disclosed in Patent Literature 2. In addition, Patent Literatures 1, 2, and 3 disclose the head mounted display for a single eye, in which the image display unit is disposed on one eye side, and Patent Literature 1 discloses the head mounted display for both eyes, in which the image display unit is disposed for each of both eyes. Furthermore, Patent Literatures 1, 2, and 3 disclose the head mounted display in which the units for supplying power and inputting/outputting signals are connected to the image display unit through the cables.

The head mounted display according to Patent Literature 1 includes an elevation angle sensor and a horizontal angle sensor in addition to the image display unit (display unit), and these angle sensors are supported by a front part of the spectacle-type frame to detect the elevation angle and the horizontal angle of the user's head.

Patent Literature 3 further discloses the head mounted display including the image display unit and the camera unit separately. The camera unit is rotatably supported by the front part, and as the attachment position of the image display unit is changed leftward or rightward, the input/output part of the camera unit, such as the cable, is rotated so that the left and right are switched. The input/output part and the image display unit are disposed on one of the left and right sides in order to connect to the control unit.

### CITATION LIST

### Patent Literature

Patent Literature 1: JP 2006-135884 A
Patent Literature 2: JP 2004-233908 A
Patent Literature 3: JP 2011-66549 A

### SUMMARY OF INVENTION

### Technical Problem

Incidentally, the head mounted displays are required to have the optimum weight balance to achieve the comfortable mount feeling, the excellent wearability, and the like so that the mounted state can be maintained easily.

However, since the image display unit is disposed in front of the eyes and, in the case of the display for single eye, disposed on one of the left and right sides, the weight of the image display unit tends to be biased on the front side and one of the left and right sides.

In regard to the head mounted display according to Patent Literature 3, the camera unit and its input/output part for the cable and the like are disposed on the front side and the input/output part comes to the same side as that of the image display unit, i.e., one of the left and right sides. In this case, the weight is remarkably biased to the front side and to one of the left and right sides.

In the case of the head mounted display in which the additional device is supported by the spectacle-type frame having the temples and the nose pads as disclosed in Patent Literatures 1 and 3, when the image display unit and moreover the sensors including the sensor for detecting the posture angle of the wearer's head and the like are provided in the front part that is disposed on the front side of the wearer's head, the load concentrates on the front side. Thus, the wearer does not feel comfortable when mounting the display and the wearability is poor in point of maintaining the mounted state. In addition, in the head mounted display according to Patent Literatures 1 and 3, the load of the cables from the units used to supply power and input/output signals and the load of the cables wired from the sensor burden the front part and concentrate on the front side remarkably.

The present invention has been made in view of the above problem in the conventional art, and an object of the present invention is to provide a head mounted display with the improved weight balance of an image display unit and an additional device that are supported by the spectacle-type frame. Thus, the wearability can be improved and the visibility of the screen can be maintained stably.

### Solution to Problem

An invention of claim 1 to solve the above problems is a head mounted display including:

an image display unit in which an image display member to be disposed in front of a wearer's eye and an image generation unit for generating an image to be displayed on the image display member are fixed to a housing, the housing containing a base end part of the image display member and the image generation unit;

a frame to be mounted on a head of the wearer, including a right front part and a left front part to be disposed on a front side of the head of the wearer, a right temple part connected to a right end of the right front part, a left temple part connected to a left end of the left front part, and supporting the image display unit;

a relay unit including a relay circuit board on which a sensor for detecting a state of the head of the wearer is mounted, and supported by the right temple part or the left temple part;

an external connection cable connected to the relay circuit board; and

an intermediate cable for connecting between the relay circuit board and the image display unit, wherein

transmission paths for signals and power between the image display unit and the unit connected to the external connection cable are formed by the external connection cable, the relay circuit board, and the intermediate cable, and transmission paths for signals and power between the sensor and the unit connected to the external connection cable are formed by the external connection cable and the relay circuit board.

An invention of claim 2 is the head mounted display according to claim 1, wherein a plurality of kinds of sensors for detecting the state of the head of the wearer is mounted on the relay circuit board.

An invention of claim 3 is the head mounted display according to claim 1 or 2, wherein the image display unit includes a proximity sensor that detects an object in front of the sensor.

An invention of claim 4 is the head mounted display according to any one of claims 1 to 3, further including a second housing incorporating an additional device and detachably attached to the right temple part or the left temple part with an attachment member formed of an elastic material, wherein:

the image display member is disposed in front of a right eye of the wearer, the image display unit is disposed in the right front part, the relay unit is disposed in the right temple part, and the second housing is disposed in the left temple part; or the image display member is disposed in front of a left eye of the wearer, the image display unit is disposed in the left front part, the relay unit is disposed in the left temple part, and the second housing is disposed in the right temple part.

Note that the aspect according to the present invention excluding the relay unit from the aspect of the present invention according to claim 7 is as below.

That is, a head mounted display includes:

an image display unit in which an image display member to be disposed in front of a wearer's right eye or left eye and an image generation unit for generating an image to be displayed on the image display member are fixed to a first housing, the first housing containing a base end part of the image display member and the image generation unit;

a frame to be mounted on a head of the wearer, including a right front part and a left front part to be disposed on a front side of the head of the wearer, a right temple part connected to a right end of the right front part, a left temple part connected to a left end of the left front part, and supporting the image display unit; and

a second housing incorporating an additional device and detachably attached to the right temple part or the left temple part with an attachment member formed of an elastic material, wherein:

the image display member is disposed in front the wearer's right eye, the image display unit is disposed in the right front part, and the second housing is disposed in the left temple part; or the image display member is disposed in front the wearer's left eye, the image display unit is disposed in the left front part, and the second housing is disposed in the right temple part.

An invention of claim 5 is the head mounted display according to claim 4, wherein the attachment member formed of the elastic material protrudes to an inside of one of the right temple part and the left temple part that corresponds to the temple part on which the second housing is mounted.

An invention of claim 6 is the head mounted display according to claim 4 or 5, wherein the attachment member formed of the elastic material is fixed to the second housing.

An invention of claim 7 is the head mounted display according to claim 4, 5, or 6, having an attachment structure where one of the right temple part and the left temple part corresponding to the temple part on which the second housing is mounted is provided with an attachment opening, and the attachment member formed of the elastic material is internally fitted to the attachment opening.

An invention of claim 8 is the head mounted display according to claim 7, wherein when the attachment member formed of the elastic material is internally fitted to the attachment opening, a retaining end part having larger width than an inner width of the attachment opening goes through the attachment opening and is disposed inside the temple part.

An invention of claim 9 is the head mounted display according to any one of claims 4 to 8, wherein the additional device is a wireless device.

An invention of claim 10 is the head mounted display according to any one of claims 4 to 9, wherein the additional device is an earphone.

An invention of claim 11 is a wearable computer including:

the head mounted display according to any one of claims 1 to 10; and

a mobile computer connected to the external connection cable of the head mounted display.

An invention of claim 12 is the wearable computer according to claim 11, wherein the mobile computer includes a wireless communication device.

An invention of claim 13 is the wearable computer according to claim 11 or 12, wherein the frame supports the image display unit on one of the right front part and the left front part, and the relay unit is supported by one of the right temple part and the left temple part that is on the same side as a side where the image display unit is supported.

### Advantageous Effects of Invention

According to the present invention, the load biased to the front side and the load biased to one of the left and right sides can be relieved. This can improve the mount feeling and reduce the feel of fatigue when wearing the head mounted display. Since the weight balance is well, the mounted state can be maintained stably during the use, and the visibility of the screen displayed by the image display unit can be maintained stably.

### BRIEF DESCRIPTION OF DRAWINGS

FIG. 1 is an external view of the entire system of a wearable computer including a head mounted display according to one embodiment of the present invention.

FIG. 2 is a perspective view viewed from the back of the head mounted display according to one embodiment of the present invention.

FIG. 3 is a perspective view viewed from the front side of the head mounted display according to one embodiment of the present invention.

FIG. 4 is a perspective view viewed from the front of the head mounted display according to one embodiment of the present invention, from which an earphone unit has been detached.

FIG. 5 is a front view of the head mounted display according to one embodiment of the present invention, in which the illustration of an external connection cable is omitted.

FIG. 6 is a top view of the head mounted display according to one embodiment of the present invention, in which the illustration of the external connection cable is omitted.

FIG. 7 is a bottom view of the head mounted display according to one embodiment of the present invention, in which the illustration of the external connection cable is omitted.

FIG. 8 is a left view of the head mounted display according to one embodiment of the present invention, in which the illustration of the external connection cable is omitted.

FIG. 9 is a right view of the head mounted display according to one embodiment of the present invention, in which the illustration of the external connection cable is omitted.

FIG. 10 is a perspective view of the earphone unit according to one embodiment of the present invention.

FIG. 11 is a front view of the earphone unit according to one embodiment of the present invention.

FIG. 12 is a front view of a portion including an image display unit of the head mounted display according to one embodiment of the present invention, from which an external cover has been detached and in which the illustration of an intermediate cable and a spectacle lens (eye-shield) is omitted.

FIG. 13 is a right view of the portion including the image display unit of the head mounted display according to one embodiment of the present invention, from which the external cover has been detached and in which the illustration of the intermediate cable, the spectacle lens (eye-shield), and the nose pad is omitted.

FIG. 14 is a bottom view of the portion including the image display unit of the head mounted display according to one embodiment of the present invention, from which the external cover has been detached and in which the illustration of the intermediate cable and the spectacle lens (eye-shield) is omitted.

FIG. 15 is a top view of the portion including the image display unit of the head mounted display according to one embodiment of the present invention, in which an upper surface of the external cover is open.

FIG. 16A is a rear view of the portion including the image display unit of the head mounted display according to one embodiment of the present invention, in which the illustration of the spectacle lens (eye-shield) is omitted.

FIG. 16B is a bottom view of the portion including the image display unit of the head mounted display according to one embodiment of the present invention, in which the illustration of the spectacle lens (eye-shield) is omitted.

FIG. 17 is a perspective view of the image display unit of the head mounted display according to one embodiment of the present invention, in which a half of the housing is detached.

FIG. 18 is a sectional view of the image display unit of the head mounted display according to one embodiment of the present invention, which is taken along an optical axis direction.

FIG. 19 is a block diagram of main circuits of the entire wearable computer including the head mounted display according to one embodiment of the present invention.

### DESCRIPTION OF EMBODIMENTS

One embodiment of the present invention will be hereinafter described with reference to the drawings. The following description is made of one embodiment of the present invention and will not limit the present invention.

### Summary of Embodiment

### (System Outline)

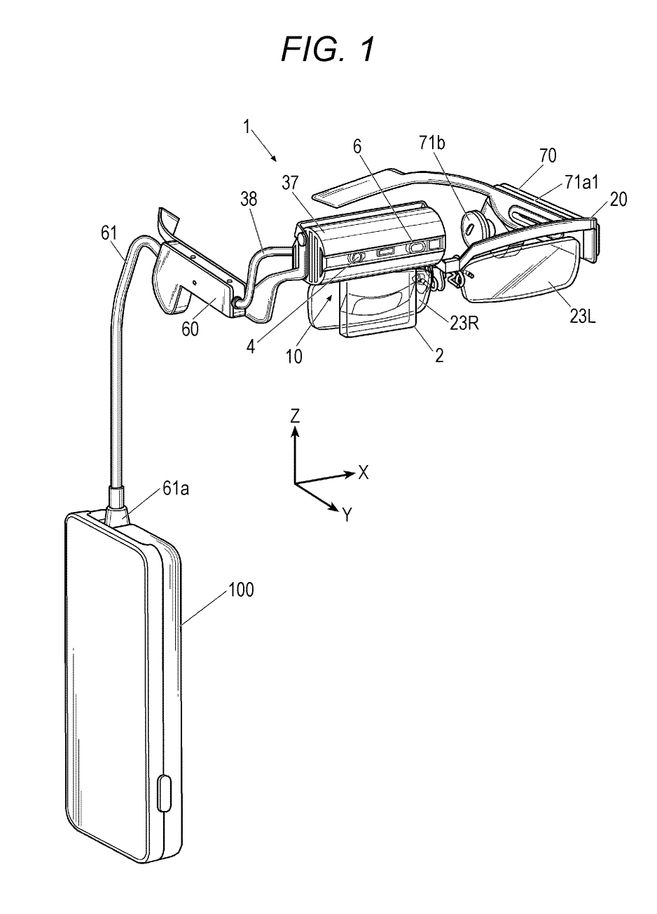

FIG. 1 is an external view of the entire system of a wearable computer including a head mounted display 1 according to this embodiment.

As illustrated in FIG. 1, the head mounted display 1 has a structure in which an image display unit 10 including an image display member 2, a relay unit 60 including a relay circuit board 81 (see FIG. 19), and an earphone unit 70 are supported by a spectacle-type frame 20 which is mounted on a wearer's head. The head mounted display 1 can be connected to a mobile computer 100 with an external connection cable 61 extending from the relay unit 60, and such connection achieves a wearable computer. An end of the external connection cable 61 includes a connector 61a connectable to the mobile computer 100. The relay unit 60 and the image display unit 10 are connected to each other with an intermediate cable 38.

Power is supplied from the mobile computer 100 to the relay unit 60 through the external connection cable 61, and the power is further supplied to the image display unit 10 through the relay unit 60 (the relay circuit board 81) and the intermediate cable 38.

Image signals are transmitted from the mobile computer 100 to the image display unit 10 through the external connection cable 61, the relay unit 60 (the relay circuit board 81), and the intermediate cable 38, and thus the image output from the mobile computer 100 is displayed to the wearer by the image display unit 10.

The image display unit 10 includes a front photographing camera 4, and an image photographed by the front photographing camera 4 is input to the mobile computer 100 through the intermediate cable 38, the relay unit 60 (the relay circuit board 81), and the external connection cable 61.

The relay unit 60 includes sensors, and detection signals from the sensors are input to the mobile computer 100 through the relay circuit board 81 and the external connection cable 61.

In addition, control signals that instruct the display setting, such as the on/off of the display, the display luminance, and the contrast of the image display unit 10, the setting of the sensitivity of the front photographing camera 4, and the like are input from the mobile computer 100 to the image display unit 10 through the external connection cable 61, the relay unit 60 (the relay circuit board 81), and the intermediate cable 38.

If necessary, control signals that instruct the setting of, for example, the on/off or the sensitivity of the sensors mounted on the relay unit 60 are input from the mobile computer 100 to the sensors mounted on the relay unit 60 through the external connection cable 61 and the relay circuit board.

The earphone unit 70 is connected to the mobile computer 100 via wireless communication, and receives sound signals from the mobile computer 100 and outputs the sound.

The mobile computer 100 includes a battery 200, a CPU 101, a ROM 102, a RAM 103, a storage device 107, and the like (see FIG. 19).

The mobile computer 100 includes a wireless communication device 104 which performs the wireless communication with the earphone unit 70, and moreover a wireless communication device 105 which can download the application program and the content by communicating with an external computer such as the Internet server (see FIG. 19). Thus, of the downloaded content or the content generated by the application program, the image can be output from the mobile computer 100 to the image display unit 10 and then displayed on the image display unit 10 and the sound can be output from the mobile computer 100 to the earphone unit 70 and thus the sound can be output.

In addition to the sensors mounted on the relay unit 60, the front photographing camera 4 and a proximity sensor 6 are provided for the image display unit 10. Detection signals from these sensors are input to the mobile computer 100. Thus, a program including the calculation based on these detection signals can be executed by the CPU 101 of the mobile computer 100.

The proximity sensor 6 of the image display unit 10 detects without contact, the presence or the motion of an object within a detection area in an proximity range in front of the sensor 6 (in a Y direction viewed from the image display unit 10). The detection signal of the presence or the motion of a user's hand is input to the mobile computer 100 through the proximity sensor 6, and the CPU of the mobile computer 100 recognizes the operation instruction corresponding to the shape of the hand or the motion of the hand (motion of the finger tip); thus a gesture input function can be achieved. Therefore, by making a particular gesture with the wearer's own hand in front of his eye where the image display unit 10 is disposed, the wearer can input the operation instruction to the mobile computer 100 and operate the mobile computer 100. Note that the object with which the wearer gives the instruction may be his hand, finger, or other object such as a stick held in his hand. In order to give an instruction to the mobile computer 100 while the position of the user's hand is recognized near his face for sure, the proximity sensor 6 preferably has a proximity range of approximately 200 mm from the detection surface of the proximity sensor 6. The proximity sensor 6 may be, for example, a known passive or active proximity sensor. The proximity sensor is generally smaller and less expensive and consumes less power than the camera. The proximity sensor is not suitable for the complicated detection, such as the detection of the object shape, but can determine when the object comes close or goes away. With the proximity sensor, the HMD can be operated by passing the hand or putting the hand over the display. Moreover, the complicated image process, which is required in recognizing the gesture by the analysis of the image photographed by the camera, is not necessary.

Here, the passive type proximity sensor includes a detection unit that detects the electromagnetic wave or the invisible light emitted from the object when the object has approached. Examples of the passive type proximity sensor include a pyroelectric sensor that detects the invisible light such as an infrared ray emitted from an approaching human body or a capacitive sensor that detects the change in electrostatic capacitance between the sensor and the approaching human body. The active type proximity sensor includes a projection unit that projects the invisible light or a sonic wave, and a detection unit that receives the invisible light or the sonic wave returning after being reflected on the object. Examples of the active type proximity sensor include an infrared ray sensor that projects the infrared ray and receives the infrared ray returning after being reflected on the object, a laser sensor that projects the laser beam and receives the laser beam returning after being reflected on the object, and an ultrasonic wave sensor that projects the ultrasonic wave and receives the ultrasonic wave returning after being reflected on the object. Note that the passive type proximity sensor is excellent in power saving property. The active type proximity sensor is excellent in detection certainty and, for example, even if the user wears a glove on his hand, the active type proximity sensor can detect the proximity. A plurality of kinds of proximity sensors can be used in combination. If the proximity sensor having a plurality of detection units disposed side by side is used, the direction of the motion of the object with respect to the detection area can be detected. In addition, based on the intensity of the output from the detection units, the direction of the object when coming close to or going away from the detection area can be detected.

(Head Mounted Display)

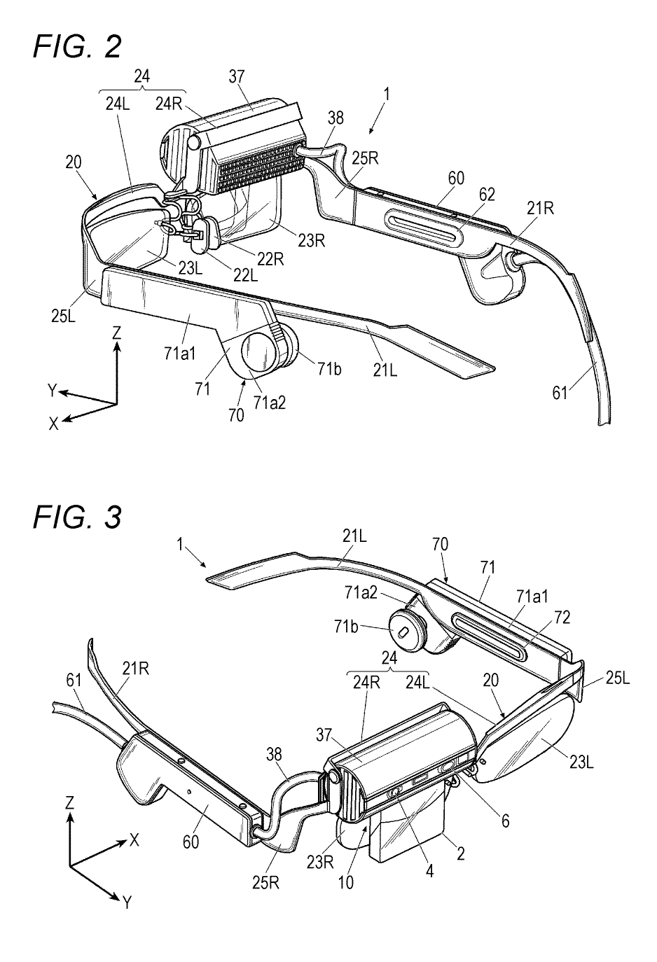

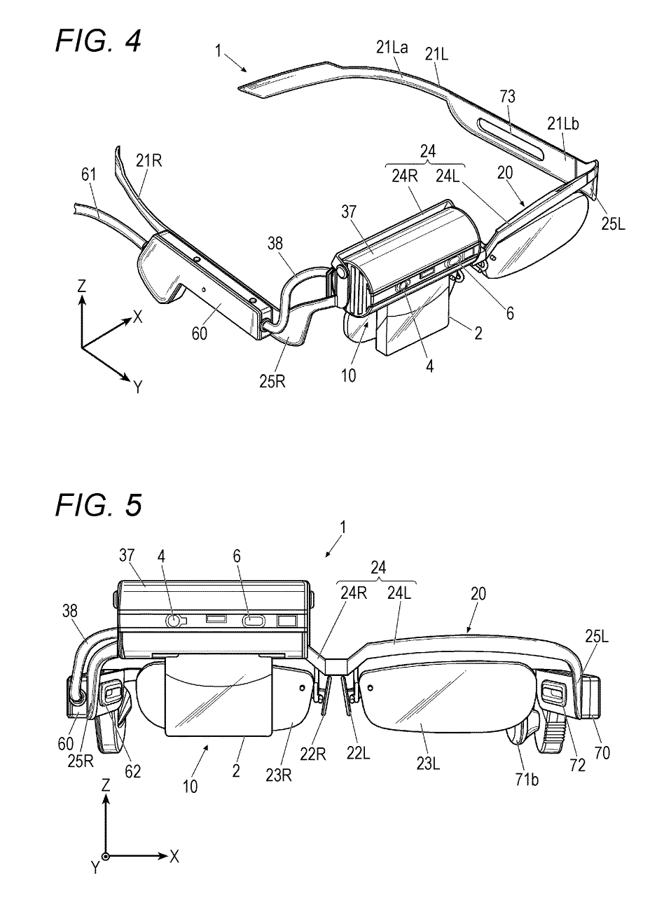

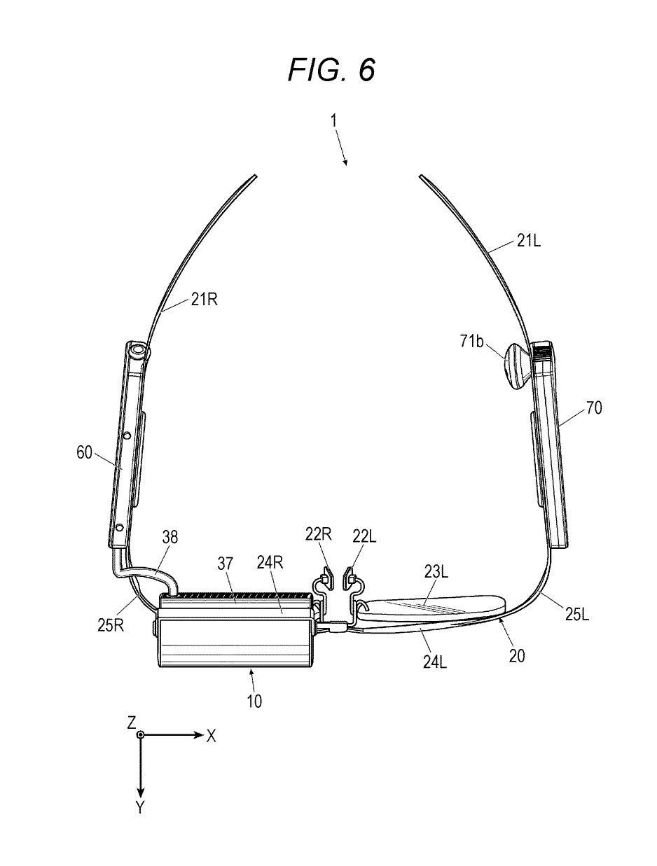

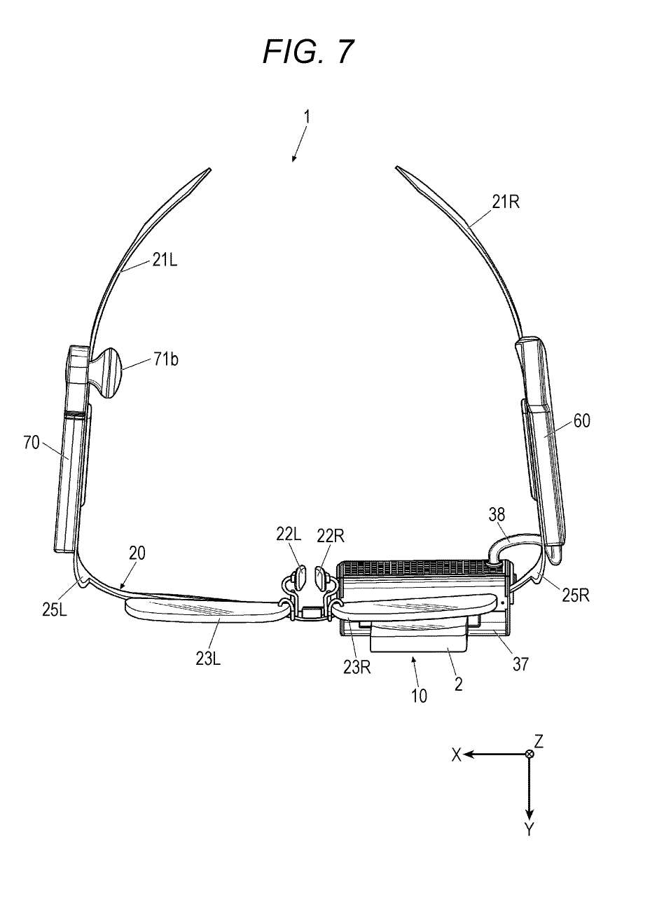

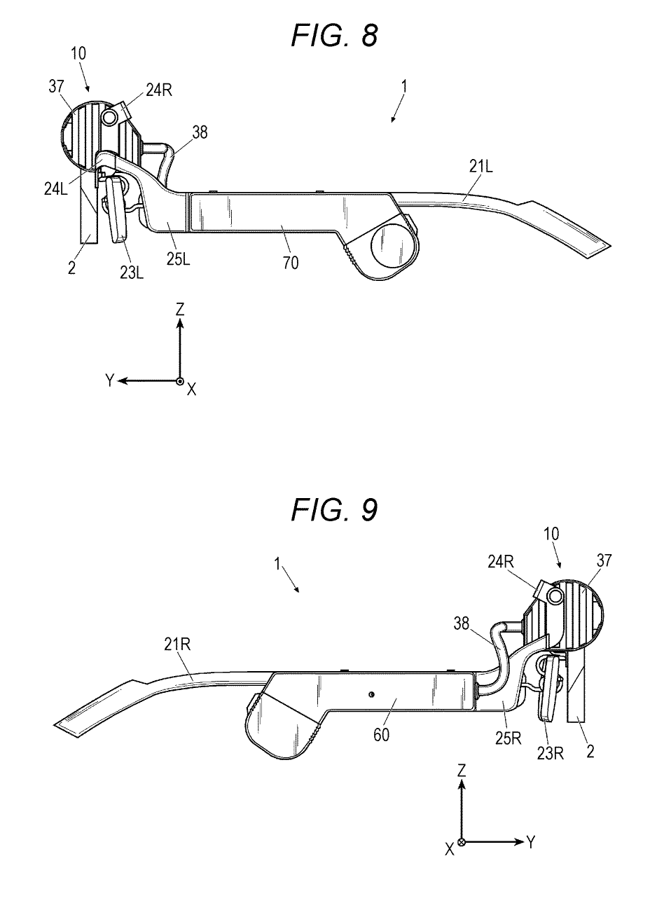

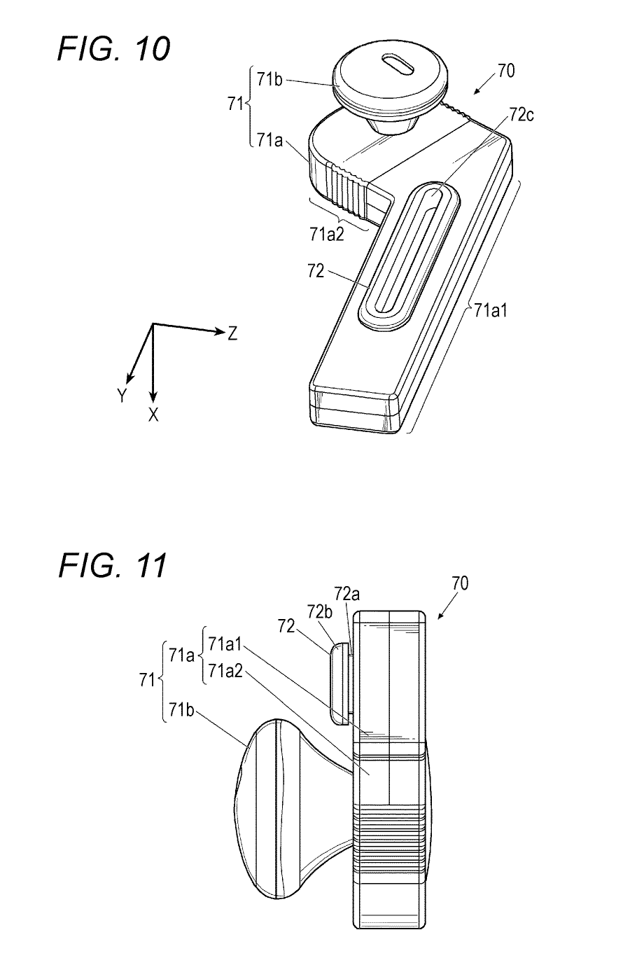

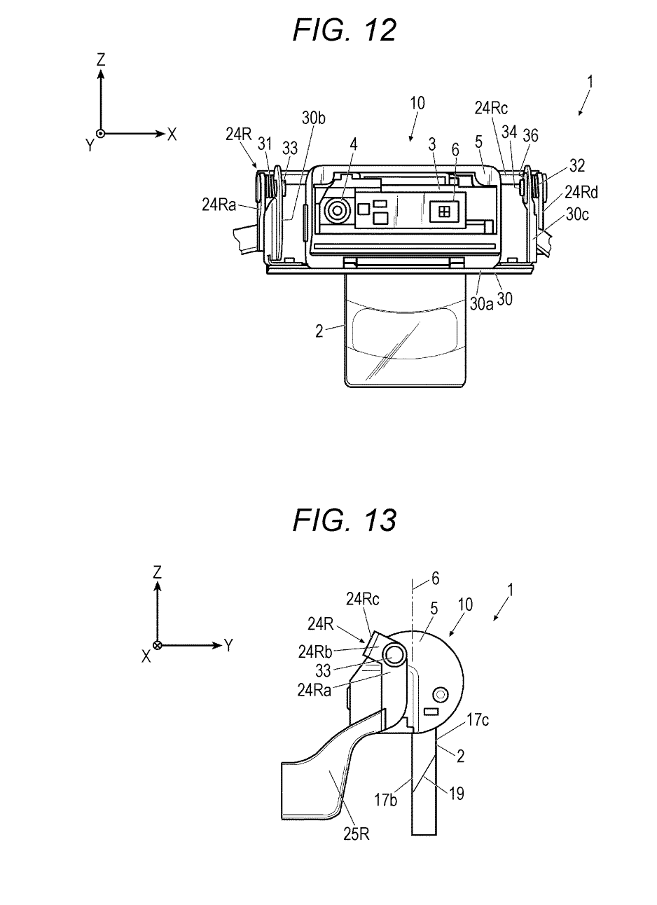

In addition, FIGS. 2 and 3 are perspective views of the head mounted display 1 viewed from the different directions. FIG. 4 illustrates the state in which the earphone unit has been detached from the state illustrated in FIG. 3. FIGS. 5 to 9 illustrate the entire head mounted display 1, and FIGS. 10 and 11 illustrate the earphone unit alone. FIGS. 12 to 18 illustrate the details of the image display unit and the support mechanism part thereof. In the drawings, the common three axes X, Y, and Z that are orthogonal to each other are shown. Based on the head of the wearer, X indicates the left-right direction (eye-width direction), Y indicates the front-back direction, and Z indicates the up-down direction.

The reference signs used in the drawings are employed in the following description.

The head mounted display 1 according to this embodiment includes: the image display unit 10 including the image display member 2, an image generation unit 3, the front photographing camera 4, a first housing 5, a proximity sensor 6, and the like; the frame 20 to be mounted on a wearer's head; a support member 30 for the image display unit 10; coil springs 31 and 32 as an energizing member; rotatable shaft members 33 and 34; fixing rings 35 and 36; and an external cover 37 as illustrated in FIGS. 12 to 17, etc.

The image display member 2 and the image generation unit 3 are fixed to the first housing 5. The first housing 5 contains a base end part 2a of the image display member 2, the image generation unit 3, and the front photographing camera 4. The image display member 2 is disposed in front of the wearer's eye, and in this embodiment, in front of the wearer's right eye. The image generation unit 3 includes a light source such as an LED, a display element such as a liquid crystal display element, a condensing lens, and the like for generating the image to be displayed on the image display member 2, and makes the generated image light enter the image display member 2. The image light having entered the image display member 2 is reflected on an optical element 19 set to an inclined surface within the image display member 2, is emitted from an inner surface 17b, and then enters the wearer's pupil (see FIG. 13). The image display member 2 constitutes a see-through type display member protruding from the first housing 5 and allowing the external light to transmit through the display member and enter the wearer's pupil. The camera 4 and the proximity sensor 6 are fixed to the image display unit 10 with the positions and directions determined in advance so that the photographing range of the camera 4 is suitable and the detection range of the proximity sensor 6 is suitable relative to the screen formed by the image display member 2.

The frame 20 supports the image display unit 10 in a manner that the image display unit 10 can be rotated through the support member 30 and the rotatable shaft members 33 and 34 extending in the eye-width direction (X direction). The image display unit 10 is supported by the support member 30 and one end of the support member 30 is connected to the rotatable shaft member 33 and the other end thereof is connected to the rotatable shaft member 34. The support member 30 is rotated relative to the frame 20 around the rotatable shaft members 33 and 34 together with the image display unit 10 (i.e., rotated around the rotatable shaft extending in the eye-width direction). Thus, the tilt angle of the image display member 2 can be adjusted. In addition, the image display unit 10 is supported so that the image display unit 10 can slide relative to the support member 30 in the eye-width direction. Thus, the position of the image display member 2 in the eye-width direction can be adjusted. The details of the rotary mechanism and the sliding mechanism will be described below.

As illustrated in FIG. 2, FIG. 3, etc., the frame 20 includes a pair of temple parts (a right temple part 21R, a left temple part 21L) to be put on the wearer's ears, a front part 24, a right corner connector 25R for connecting between the right temple part 21R and the front part 24 (the right end of the right front part 24R), and a left corner connector 25L for connecting between the left temple part 21L and the front part 24 (the left end of the left front part 24L). This enables the wearer to wear the present head mounted display 1 on his head just like the spectacles. Note that the frame 20 may be bent by using hinges to connect between the right temple part 21R and the right corner connector 25R and between the left temple part 21L and the left corner connector 25L, or the temple part and the corner connector may be connected integrally or molded integrally to provide a hinge-less frame.

To the front part 24, nose pads 22L and 22R to be set on the wearer's nose, and spectacle lenses or eye-shields 23L and 23R are connected and fixed.

The front part 24 is disposed on the front side of the wearer's head. Of the front part 24, the right half corresponds to the right front part 24R and the left half corresponds to the left front part 24L.

In the present embodiment, the image display member 2 is disposed in front of the wearer's right eye and the image display unit 10 is disposed in the right front part 24R and the earphone unit 70 is disposed in the left temple part 21L. The relay unit 60 is disposed in the right temple part 21R.

In an embodiment of displaying an image for the left eye, the right and left in this embodiment is set opposite: the image display member 2 is disposed in front of the wearer's left eye and the image display unit 10 is disposed in the left front part 24L, and the earphone unit 70 is disposed in the right temple part 21R. In this case, the relay unit 60 is disposed in the left temple part 21L.

(Earphone Unit)

As illustrated in FIGS. 10 and 11, the earphone unit 70 includes a second housing 71. That is, the additional device incorporated in the second housing 71 is a wireless earphone in this embodiment. The second housing 71 is formed by a board container 71a and an inner ear part 71b. The board container 71a contains a communication circuit that receives a sound signal, a driving circuit that amplifies the received sound and drives a vibration unit included in the inner ear part 71b, a battery to supply power to these circuits, and the like.

The board container 71a includes a front part 71a1 formed long along the left temple part 21L, and a rear part 71a2 that protrudes downward continuing from the rear end of the front part 71a1 to support the inner ear part 71b. As illustrated in FIGS. 1 to 3, etc., the front part 71a1 can be disposed neatly along the left temple part 21L and moreover the inner ear part 71b can be disposed in the ear hole of the wearer without causing the interference between the inner ear part 71b and the left temple part 21L.

The second housing 71 is detachably attached to the left temple part 21L with an attachment member 72 formed of an elastic material.

The elastic material used for the attachment member 72 is preferably elastomer. The attachment member 72 is fixed to an inner surface of the front part 71a1 of the board container 71a, and is formed long in the front-back direction (Y direction) in a manner similar to the front part 71a1.

On the other hand, as illustrated in FIG. 4, the left temple part 21L includes an ear hook part 21La and a front part 21Lb. The front part 21Lb is more on the front side than the ear hook part 21La and will be in contact with the side surface of the wearer's head. The front part 21Lb is provided with an attachment opening 73. The attachment member 72 and the attachment opening 73 are used to have the earphone unit 70 detachably attached to the left temple part 21L and are both formed long in the front-back direction (Y direction). The attachment member 72 and the attachment opening 73 are formed long in the front-back direction (Y direction) in this manner in order to fix the earphone unit 70 to the left temple part 21L firmly without backlash even when the number of attachment members 72 is one and the number of attachment openings 73 is one. When the number of attachment members 72 is one, the single attachment member 72 is bonded to the second housing 71 in a larger area so that the separation of the attachment member 72 from the second housing 71 can be prevented. In addition, the attachment member 72 is in contact with the side surface of the wearer's head and functions as the nonslip material and the cushion material. Thus, these functions can be achieved effectively.

As illustrated in FIG. 11, the attachment member 72 includes a base part 72a to be bonded to the second housing 71, and a retaining end part 72b formed at an end of the base part 72a opposite to the end thereof to be bonded to the second housing 71.

The outer periphery of the base part 72a substantially coincides with the inner periphery of the attachment opening 73. The retaining end part 72b is formed a little larger than the inner periphery of the attachment opening 73. That is, the retaining end part 72b has larger width than the inner width of the attachment opening 73 both in the Y direction and in the Z direction.

When the retaining end part 72b is disposed in accordance with the attachment opening 73 and the earphone unit 70 is pressed against the left temple part 21L, the retaining end part 72b comes to the inside of the left temple part 21L through the attachment opening 73 and thus, the attachment structure in which the attachment member 72 is internally fitted to the attachment opening 73 is achieved.

With such an attachment structure, the earphone unit 70 is mounted on the left temple part 21L and when the present head mounted display 1 is mounted on the wearer's head, the retaining end part 72b of the attachment member 72 protrudes to the inside of the left temple part 21L to be brought into contact with the side surface of the wearer's head. Thus, the attachment member 72 formed of the elastic material functions as the nonslip material and the cushion material. Therefore, it is possible to prevent the head mounted display 1 from being displaced from the wearer's head or falling off from the head and moreover improve the wearability. In particular, in regard to the wearability, the head mounted display 1 is displaced less easily and the attachment member 72 can be set to the wearer's head softly, so that the wearer can feel comfortable in wearing the display for a long time.

By engaging the attachment member 72 formed of the elastic material to the temple part as above, the earphone unit 70 can be attached/detached easily without a complicated structure such as screwing.

In order to easily deform the retaining end part 72b, specifically reduce the diameter thereof in the attachment/detachment, the attachment member 72 is provided with a hole 72c that opens inside the retaining end part 72b as illustrated in FIG. 10.

In order to reduce the diameter of the retaining end part 72b so as to easily pass through the attachment opening 73 in the attachment and to enable the retaining end part 72b in contact with the wearer's head gently, the corners of the outer periphery of the retaining end part 72b in the inserting direction are rounded as illustrated in FIG. 11.

The attachment member 72 is fixed to the second housing 5, and always accompanies the earphone unit 70 during the use including the attachment/detachment. This prevents the loss of the attachment member 72 and moreover since the attachment/detachment mechanism is not provided between the second housing 71 and the attachment member 72, the second housing 71 is not complicated.

The additional device incorporated in the second housing 71 is the wireless earphone in this embodiment but may be a camera or various kinds of sensors, for example.

(Relay Unit)

The relay unit 60 has the outer shape substantially the same as that of the board container 71a of the earphone unit 70. To attach the relay unit 60 to the right temple part 21R, the same attachment/detachment mechanism as that used to attach the earphone unit 70 to the left temple part 21L is employed. An attachment member 62 of the relay unit 60 is illustrated in FIG. 2, etc. The attachment member 62 also functions as the nonslip material and the cushion material. Since the attachment members 62 and 72 are in contact with the wearer's head from both sides, the wearability is improved remarkably.

The relay circuit board 81 of the relay unit 60 is provided with a plurality of kinds of sensors to detect the state of the wearer's head. In this embodiment, a gyro (angular speed sensor) 82 and an electronic compass (a geomagnetism sensor) 83 are provided as the sensors to detect the state of the wearer's head (see FIG. 19). Since the plurality of kinds of sensors is mounted to detect the state of the wearer's head, the state of the wearer's head can be detected more accurately. Another sensor such as an acceleration sensor or a pressure sensor (to detect the pressure at the position of the head) may be mounted. The sensors mainly mounted are for detecting the position, the direction, or the motion of the wearer's head. When the sensors that are required to be set near the head for the purpose of detecting the signals from the head, such as the electroencephalography or the infrared ray, are mounted on the relay circuit board, these sensors can be disposed near the wearer's head and in this case, the load on the front side of the head is not increased.

The relay unit 60 incorporates a microphone 84, and the microphone 84 is mounted on the relay circuit board 81 (see FIG. 19). The microphone 84 generates an output signal in accordance with the user's sound, the CPU 101 of the mobile computer 100 analyzes the user's sound, and controls the units in accordance with the user's intent.

With an unshown wiring pattern on the relay circuit board 81, the transmission paths for power and signals in the relay unit 60 are constructed. The transmission paths for the power on the relay circuit board 81 include the transmission path for supplying power to the image display unit 10. The transmission paths for the signals on the relay circuit board 81 include the transmission paths for the detection signals of the proximity sensor 6 and the photographed image data of the front photographing camera 4 of the image display unit 10.

To the relay circuit board 81 of the relay unit 60, an end of the external connection cable 61 and an end of the intermediate cable 38 are connected by soldering or the like. Therefore, even if the relay unit 60 is detached from the right temple part 21R by using the elasticity of the attachment member 62, the external connection cable 61, the relay unit 60, the intermediate cable 38, the image display unit 10, and the frame 20 remain connected. In order to facilitate the attachment/detachment by separating the relay unit 60 from the frame 20 like the earphone unit 70, the end of the intermediate cable 38 may be provided with a connector but by not providing such a connector, it is possible to avoid the complication, the size increase, and weight increase of the head mounted display 1. Moreover, since the relay unit 60 is supported by one of the right temple part 21R and the left temple part 21L on the same side as the side where the image display unit 10 is supported, the increase in length of the intermediate cable 38 can be prevented.

(Circuit Block)

FIG. 19 is a block diagram of main circuits of the wearable computer. On a circuit board 90 set in the image display unit 10, a display panel driver 91 as a driving circuit for driving a display element 14, a light source driving circuit 92 for driving a light source 11, an illuminance sensor 93 for detecting the brightness of the surrounding environment, and a power source circuit 203 are provided in addition to the camera 4 and the proximity sensor 6. Based on the output from the illuminance sensor 93, the light source driving circuit 92 and the display panel driver 91 are controlled to adjust the luminance of the image to be displayed on the image display member 2; thus, the user can easily see the image. On the relay circuit board 81 set in the relay unit 60, an EEPROM 85 for storing various pieces of information and a power source circuit 202 are provided in addition to the gyro 82, the electronic compass 83, and the microphone 84. The power source circuit 202 supplies power to the units on the relay circuit board 81 and relays the power supply to the image display unit 10 in a subsequent stage. The circuit board 110 in the mobile computer 100 includes the CPU 101 that controls the whole, the ROM 102 and the RAM 103 that store the programs and various pieces of information, the wireless communication devices 104 and 105, an operation unit 106 that executes various instructions including the on/off of the power source of the entire system, the storage device 107 such as a flash memory, an interface 108 for connecting with the external device, and a power source circuit 201 to be connected to the detachable battery 200. The signal transmission path where the control signals and various pieces of information are exchanged and the power transmission path where the power is supplied are connected to the external connection cable 61 through the connector 61a. The signal transmission paths and the power transmission paths between the circuit board 90 of the image display unit 10 and the relay circuit board 81 of the relay unit 60 are connected to each other through the intermediate cable 38. The earphone unit 70 includes a wireless earphone device 120 and a sub-battery 121 as the power source that supplies power to the wireless earphone device 120. As illustrated in FIG. 19, in this example, the user operates the wearable computer through the noncontact user interface such as the proximity sensor 6 or the microphone 84. Such operation suppresses the risk of displacing the head mounted display but an operation unit through which the operation is carried out in the range where the excess displacement does not occur may be provided for the relay unit 60, the image display unit 10, the frame 20 of the head mounted display 1, or the like. For example, a touch sensor may be provided to enable the touch operation or swipe operation, or a key may be disposed at such a position that the user can press the key with his finger while holding the additional device with another finger.

As described above, the transmission paths for signals and power between the image display unit 10 and the unit (the mobile computer 100) connected to the external connection cable 61 may be formed by the external connection cable 61, the relay circuit board 81 in the relay unit 60, and the intermediate cable 38 and the transmission paths for signals and power between the sensors in the relay unit 60 and the unit (the mobile computer 100) connected to the external connection cable 61 maybe formed by the external connection cable 61 and the relay circuit board 81.

(Operation Effect and Others)

According to the present embodiment as described above, the image display unit 10 is disposed on the front side of the head mounted display 1 and on the other hand, the relay unit 60 is disposed in the temple part 21 (in this example, the right temple part 21R) on the back side relative to the image display unit 10. Thus, the weight balance on the front side and the back side due to the image display unit 10 and the additional device supported by the spectacle-type frame 20 can be improved.

That is, by diffusing the load to the temple part while the sensors are mounted to enhance the functions, the load biased to the front side can be relieved. This can improve the mount feeling and reduce the fatigue during the wearing.

The external connection cable 61 is provided extending from the rear end of the relay unit 60 to the rear side. Thus, the load of the external connection cable 61 is not applied to the front part 24 and the load biased to the front side can be relieved.

Since the weight balance is well, the mounted state can be maintained stably during the use and the visibility of the screen displayed by the image display unit 10 can be maintained stably.

According to the present embodiment, the image display unit 10 is disposed on the front side of one of the left and right sides (in this embodiment, in the right front) of the head mounted display 1. On the other hand, the additional device and the housing thereof (the earphone unit 70) are disposed in the left temple part 21L that is farther from the image display unit 10; thus, the weight balance of the image display unit 10 and the additional device supported by the spectacle-type frame 20 between the front and back sides and the left and right sides can be improved.

Moreover, the additional device and the housing thereof (the earphone unit 70) are disposed in one of the left and right temple parts and the relay unit 60 is disposed in the other temple part; thus, the weight balance on the left and right sides can be improved.

That is, the load biased to the front side or to one of the left and right sides can be relieved. Thus, the mount feeling can be improved and the fatigue during the wearing can be reduced.

Since the weight balance is well, the mounted state can be maintained stably during the use and the visibility of the screen displayed by the image display unit 10 can be maintained stably.

When the head mounted display 1 is attached to or detached from the head, the second housing 71 is attached to or detached from the temple part 21L, or the head mounted display 1 is used or handled, a force may be applied to the temple part 21L to cause the deformation. However, since the attachment member 72 formed of the elastic material is deformed to absorb such deformation, the application of dynamic burden on the second housing 71 can be suppressed. Thus, the second housing 71 can remain attached to the temple part 21L without causing the damage, the operation error, or the like of the additional device incorporated in the second housing 71 due to the deformation of the temple part 21L. In regard to the relay unit 60, the similar effect is also achieved by the deformation of the attachment member 62 formed of the elastic material.

In addition, by the attaching/detaching function of the second housing 71, the function of the head mounted display 1 can be enhanced or the function/performance thereof can be changed, or the external appearance design of the head mounted display 1 can be changed. For example, the function can be enhanced by attaching the earphone unit 70 to the head mounted display 1, the function can be changed by replacing the earphone unit 70 with a GPS unit, the performance can be changed by replacing the earphone unit 70 with another earphone unit having the different performance, and the function/performance can be enhanced by replacing the earphone unit 70 with a unit having the GPS function and the earphone function. In addition, a unit with a different color or shape can be used or a unit with smaller thickness and weight can be used, and in the occurrence of a failure, the unit can be replaced with the same type of unit. The relay unit 60 is similarly replaceable by using a connector for the intermediate cable 38.

According to this embodiment, the mobile computer 100 includes the wireless communication device. Thus, the system capable of wireless communication can be formed without increasing the weight of the head mounted display 1, i.e., the unit to be mounted on the wearer's head.

The aforementioned gyro and electronic compass are relatively large and heavy, while the camera and the proximity sensor are relatively small and lightweight. According to this embodiment, the gyro and the electronic compass are mounted on the relay unit 60 of the temple part and the front camera and the proximity sensor are mounted on the image display unit 10 in the front part 24; thus, the load biased to the front side can be relieved and the front-back weight balance is well. Note that the proximity sensor 6 mounted on the image display unit 10 can be omitted and by using the front photographing camera 4 instead, the gesture input function may be achieved.

[Details of Other Units]

Complementary description is made of the details of the units.

### (Rotary Mechanism)

As illustrated in FIGS. 12 to 14, the right front part 24R includes flange parts 24Ra and 24Rb extending from an installation portion of the rotatable shaft member 33 in the radial direction thereof, flange parts 24Rd and 24Re extending from an installation portion of the rotatable shaft member 34 in the radial direction thereof, and a crossing part 24Rc connecting the flange part 24Rb and the flange part 24Re on both sides of the first housing 5 and crossing the first housing 5 in the eye-width direction (X direction).

On the other hand, the support member 30 extends in the eye-width direction (X direction) and includes a bottom part 30a on which the image display unit 10 is mounted, and arm parts 30b and 30c extending from opposite ends of the bottom part 30a on a YZ plane.

On each side of the image display unit 10, the rotatable shaft member 33 (34) is inserted into the hole provided for the flange part 24Ra (24Rd), the coil spring 31 (32), and the hole provided for the arm part 30b (30c), and an end of the rotatable shaft member 33 (34) is fitted to the fixing ring 35 (36) and fixed to the fixing ring 35 (36); thus, the support member 30 supporting the image display unit 10 is rotatably connected to the frame 20.

With the above structure, one of a pair of rotatable shaft members 33 and 34 is disposed on one of the opposite side surfaces of the first housing 5 in the eye-width direction (X direction) and the other of the pair is disposed on the other side surface.

The rotatable shaft members 33 and 34 face each other at the side surfaces of the first housing 5 in the eye-width direction (X direction). That is, the center shaft of the rotation is positioned through the first housing 5. Although the center shaft of the rotation is positioned through the first housing 5, the rotatable shaft member 33 on one side and the rotatable shaft member 34 on the other side are connected while avoiding the first housing 5 due to the flange part 24Rb, the flange part 24Re, and the crossing part 24Rc. Since the center shaft of the rotation is positioned through the first housing 5, the space where the image display unit 10 moves along with the rotation (especially the first housing 5 part) can be made smaller and the convenience and the compactness can be maintained.

In addition, as illustrated in FIG. 13, the center shafts of the rotatable shaft members 33 and 34 are disposed more on the wearer's side than a plane 6 where the image light emission surface 17b of the image display member 2 exists. Thus, since the center shafts of the rotatable shaft members 33 and 34 are positioned closer to the wearer's pupil, the change in tilt angle to the wearer's visual line is gentle for the amount of movement of the image display member 2 in the tilt angle adjustment. Therefore, the tilt angle can be minutely adjusted easily.

When the image display unit 10 is rotated, the image display unit 10 side and the frame 20 side of the image display unit 10 are energized to each other in the axial direction (X direction) of the rotatable shaft members 33 and 34 with the coil springs 31 and 32 in the above structure. Thus, the friction resistance occurs in the rotation and the angle of the rotation can be maintained. That is to say, the image display unit 10 is supported by the frame 20 so that the image display unit 10 can be held at any angle position by the frame 20.

In this embodiment, the coil spring 31 as the energizing member is provided at one side of the image display unit 10 in the eye-width direction (X direction) and the coil spring 32 as the energizing member is provided at the other side thereof. Thus, when the image display unit 10 is pressed from both sides (directly the support member 30 is pressed) with the coil springs 31 and 32 on the opposite sides, the friction resistance is generated.

More specifically, one end of the rotatable shaft member 33 (34) is connected to the flange part 24Ra (24Rb) of the frame 20 and the other end of the rotatable shaft member 33 (34) is connected to the arm part 30b (30c) of the support member 30. By pressing the side surface of the support member 30 in the eye-width direction (X direction), i.e., the surface of the arm part 30b (30c) with the coil spring 31 (32), the friction resistance is generated.

As described above, the tilt angle of the image display member can be minutely adjusted and the adjusted angle can be maintained stably. Adjusting the tilt angle near the eye produces the effect similar to the effect obtained when the position of the display screen of the image display member 2 is adjusted in the substantially vertical direction. In addition, the position in the front-back direction (depth direction) can be adjusted for a certain degree. Therefore, with the tilt adjusting mechanism, the minute adjustment in the up-down direction and the front-back direction becomes possible. With the angle adjustment of the image display member 2, the camera 4 and the proximity sensor 6 are rotated at the same time; thus, the rotation operation will not cause the displacement between the camera 4 and the proximity sensor 6, and the image display member 2.

(External Cover)

As illustrated in FIG. 15, the external cover 37 is fixed to the support member 30, and is rotated together with the image display unit 10 and the support member 30. The external cover 37 covers the support member 30 and the first housing 5, and exposes the image display member 2. Since the portion to be rotated is covered with the external cover 37, the entry of foreign substances to interrupt the tilt angle adjusting function can be suppressed.

(Intermediate Cable)

The intermediate cable 38 is used to connect the image display unit 10 to the relay unit 60.

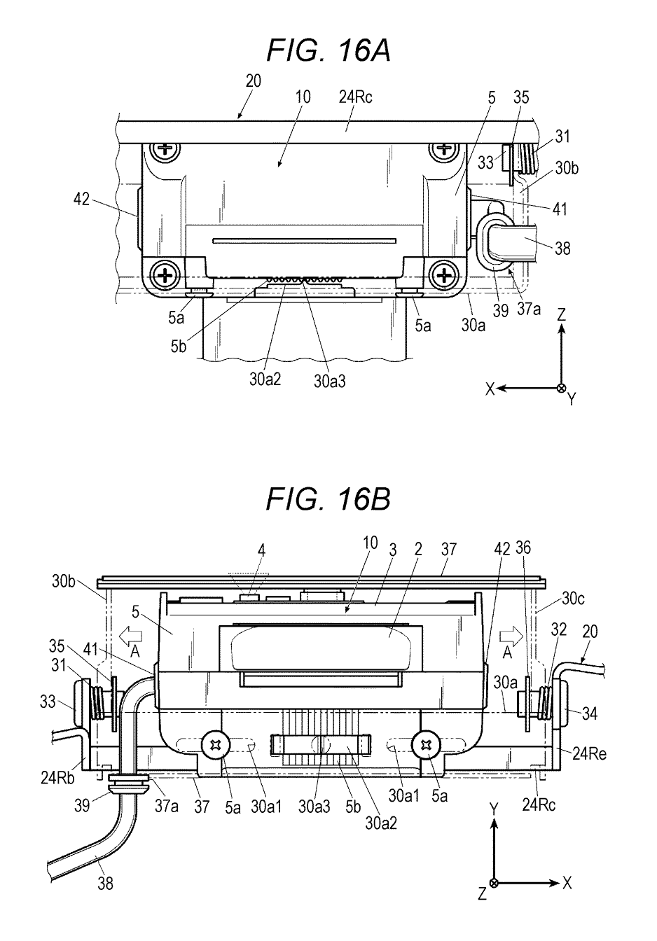

As illustrated in FIGS. 16A and 16B, the intermediate cable 38 extends from the first housing 5 through the hole 37a provided for the external cover 37, and is movable relative to the hole 37a. In this embodiment, the space between the intermediate cable 38 and the hole 37a is filled with a bush 39 fitted to the hole 37a, so that the dust-proof property of the external cover 37 is secured, and the material and the size are selected so that the intermediate cable 38 slides relative to the bush 39.

(Sliding Mechanism)

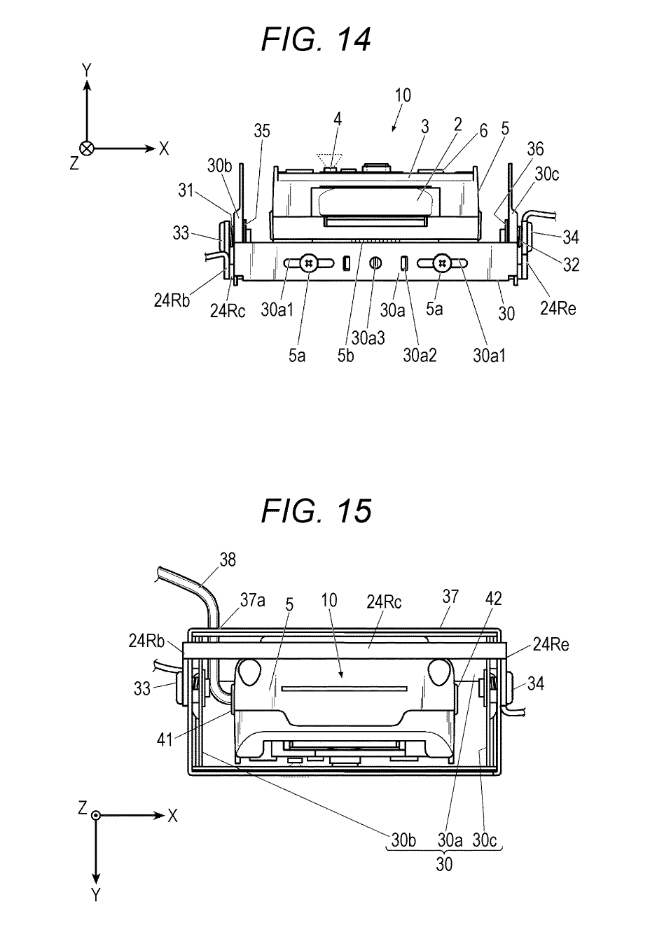

As illustrated in FIGS. 14, 16A, and 16B, the support member 30 supports the image display unit 10 through the sliding mechanism in a manner that the image display unit 10 can move in the eye-width direction (X direction).

The sliding mechanism includes an engaging mechanism for keeping the sliding operation at predetermined pitches, and a sliding guide mechanism is formed by guide holes 30a1 and 30a1 provided at the bottom part 30a of the support member 30, and guide pins 5a and 5a inserted into the guide holes 30a1 and 30a1 and having an end fixed to the first housing 5. The engaging mechanism is formed by a holding groove band 5b provided for a surface of the first housing 5 opposite to the bottom part 30a, and a plate spring 30a2 having a protrusion 30a3 to be fitted to the groove formed in the holding groove band 5b.

The guide holes 30a1 and 30a1 extend long in the X direction, and the space is formed between the first housing 5 and the arm parts 30b and 30c of the support member 30, and the guide pins 5a and 5a are loosely fitted to the guide holes 30a1 and 30a1; thus, the image display unit 10 is movable in the eye-width direction (X direction) relative to the support member 30, the external cover 37, and the frame 20 as indicated by arrows A in FIG. 16B.

As illustrated in FIGS. 16A and 16B, the holding groove band 5b includes a plurality of grooves cut in the Y direction and formed continuously at predetermined pitches in the X direction, and the plate spring 30a2 exerts the elastic force so that its protrusion 30a3 is pressed against the holding groove band 5b. This causes the protrusion 30a3 to be fitted into each groove of the holding groove band 5b, and the sliding operation of the image display unit 10 can be kept at predetermined pitches.

As described above, even if the image display unit 10 slides relative to the external cover 37 and moreover the external cover 37 moves relative to the frame 20 as the tilt angle is adjusted, such operations can be performed smoothly without applying the dynamic burden to the intermediate cable 38 because the intermediate cable 38 is movable to be taken in and out of the external cover 37.

(Dust-Proof/Water-Proof Structure of Image Display Unit)

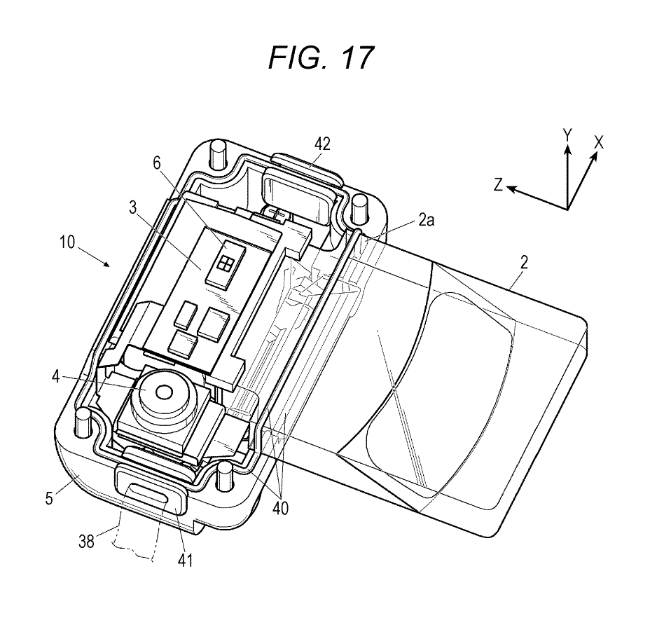

In addition, the dust-proof/water proof properties of the first housing 5 are secured by a sealing member 40 and moreover by a bush 41 and a cap 42 illustrated in FIG. 17.

The first housing 5 is separated into halves, and the sealing member 40 surrounds the periphery of a separation surface of the first housing 5 and moreover the periphery surrounding the base end part 2a of the image display member 2. That is, the sealing member 40 has a structure in which an annular portion to which the image display member 2 is inserted is connected to the portion held between the two halves of the first housing 5. The separation surfaces of the first housing 5, and the first housing 5 and the base end part 2a of the image display member 2 are sealed.

The bush 41 is fitted to the hole of the first housing 5 for drawing out the intermediate cable, and seals between the intermediate cable 38 and the first housing 5. Since the intermediate cable 38 is not needed to be movable relative to the bush 41, the material and the size are selected so that the bush 41 tightly fastens the intermediate cable 38.

The cap 42 attached to the side opposite to the side of the bush 41 is a lid member that covers the cable drawing hole when the image display unit 10 is attached to the left side. Thus, the image display unit 10 can be attached either to the right side or to the left side. The cap 42 corresponds to the filling of the hole of the bush 41.

As described above, the dust-proof/water-proof properties of the first housing 5 are secured and the entry of the liquid such as water and the gas such as water vapor into the image generation unit 3, the front photographing camera 4, and the like in the first housing 5 can be prevented.

(Display Function of Image Display Unit)

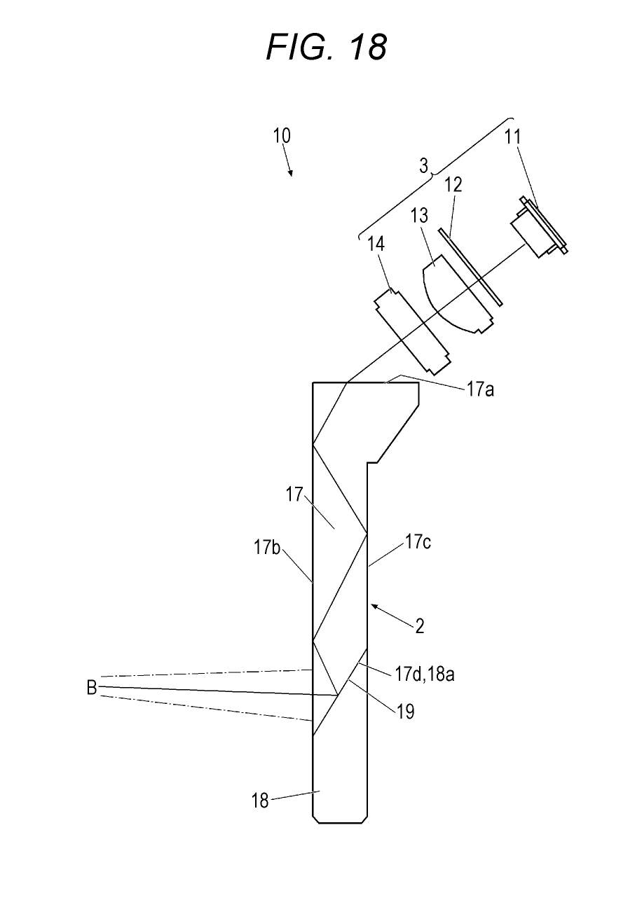

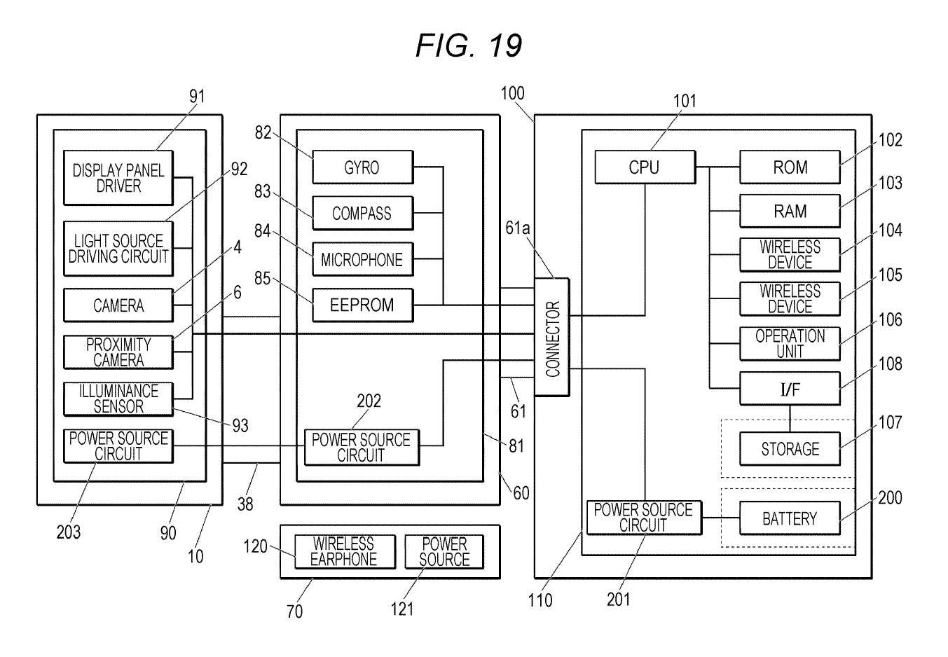

FIG. 18 is a sectional view illustrating a schematic structure of the image display unit 10.

The image display unit 10 includes the light source 11, a unidirectional diffusing plate 12, a condensing lens 13, the display element 14, and the image display member 2. The image display member 2 includes a see-through type display member.

The light source 11 is to illuminate the display element 14, and includes, for example, RGB-integrated LEDs emitting light with three wavelength ranges of 462±12 nm (B light), 525±17 nm (G light), and 635±11 nm (R light), which correspond to the peak wavelength and the wavelength width at half intensity. In this manner, since the light source 11 emits the light with the predetermined wavelength widths, the image light obtained by illuminating the display element 14 can have the predetermined wavelength widths and when the image light is diffracted by a hologram optical element 19, the wearer can observe the image in the entire range of the viewing angle at the position of the pupil B. The peak wavelength of each color of the light source 11 is set near the peak wavelength of the diffraction efficiency of the hologram optical element 19, so that the light use efficiency is improved.

The light source 11 can be formed at lower cost because of using the LEDs emitting the RGB light, and moreover the display element 14 can display the color image when illuminated by the light source 11 and in this case, the color image can be provided to the wearer. Since the emission wavelength width of each LED element of RGB is narrow, using a plurality of such LED elements can achieve the high color reproducing property and the bright image display.

The display element 14 is to display an image by modulating the emission light from the light source 11 in accordance with the image data, and includes a transmissive liquid crystal display element having the pixels, where the light is transmitted, arranged in matrix. Note that the display element 14 may be the reflective type.

An ocular prism 17 totally reflects the image light from the display element 14, which is incident through a base end surface 17a, on the inner surface 17b and an outer surface 17c which are parallel to each other, and guides the totally reflected light to the pupil of the wearer through the hologram optical element 19 and on the other hand, the ocular prism 17 transmits the external light and guides the transmitted light to the pupil of the wearer's eye. The ocular prism 17 and a deflecting prism 18 are formed of, for example, acrylic resin.

The ocular prism 17 and the deflecting prism 18 are bonded with adhesive in a state that the hologram optical element 19 is held between inclined surfaces 17d and 18a that are inclined relative to the inner surface 17b and the outer surface 17c.

The deflecting prism 18 is bonded to the ocular prism 17 to be integrated, so that the prisms serve as the approximately parallel flat plate. By boding this deflecting prism 18 to the ocular prism 17, the distortion in the external world image to be observed by the wearer through the image display member 2 can be prevented.

That is, for example, if the deflecting prism 18 is not bonded to the ocular prism 17, the external light is refracted when passing the inclined surface 17d of the ocular prism 17, so that the external world image to be observed by the wearer through the ocular prism 17 is distorted. However, when the deflecting prism 18 having the inclined surface 18a complementary to the ocular prism 17 is bonded to the ocular prism 17 to form the integral approximately parallel flat plate, the refraction of external light when passing the inclined surfaces 17d and 18a (the hologram optical element 19) can be canceled by the deflecting prism 18. As a result, the distortion in the external world image to be observed by the wearer through the image display member 2 can be prevented.

Note that those who normally wear glasses can also observe the image without any problem by wearing correction spectacle lenses between the image display member 2 and the pupil of the wearer.

The hologram optical element 19 is a volume phase type reflective hologram that diffracts and reflects the image light (the light with the wavelengths corresponding to the three primary light) emitted from the display element 14, and guides the light to the pupil B, and magnifies the image displayed on the display element 14 and guides the magnified image to the pupil of the wearer as a virtual image. The hologram optical element 19 is fabricated so as to, for example, diffract (reflect) the light with three wavelength ranges of 465±5 nm (B light), 521±5 nm (G light), and 634±5 nm (R light), which correspond to the peak wavelength of the diffraction efficiency and the wavelength width at half diffraction efficiency. Here, the peak wavelength of the diffraction efficiency is the wavelength at which the diffraction efficiency is the maximum, and the wavelength width at the half diffraction efficiency is the wavelength width at which the diffraction efficiency is a half of the diffraction efficiency peak.

The reflective hologram optical element 19 has the high wavelength selectivity, and diffracts and reflects only the light with the wavelength in the aforementioned wavelength range (near the exposure wavelength). Therefore, the external light with the wavelength other than the wavelength to be diffracted and reflected passes through the hologram optical element 19, and thus the high external light transmissivity can be achieved.

The light emitted from the light source 11 is diffused by the unidirectional diffusing plate 12, condensed by the condensing lens 13, and then enters the display element 14. The light having entered the display element 14 is modulated for each pixel on the basis of the image data and emitted as the image light. That is, the color image is displayed on the display element 14.

The image light from the display element 14 enters the ocular prism 17 from the base end surface 17a and is totally reflected on the inner surface 17b and the outer surface 17c a plurality of times, and enters the hologram optical element 19. The light having entered the hologram optical element 19 is reflected thereby and reaches the pupil B through the inner surface 17b. At the position of the pupil B, the wearer can observe the magnified virtual image of the image displayed on the display element 14.

On the other hand, the ocular prism 17, the deflecting prism 18, and the hologram optical element 19 transmit most of the external light and therefore the wearer can observe the external world image therethrough. Therefore, the virtual image of the image displayed on the display element 14 can be observed overlapped on a part of the external world image.

As described above, the wearer can observe the external world image and the image provided by the display element 14 at the same time through the hologram optical element 19.

Incidentally, the hologram optical element 19 has the wavelength selectivity and enables the viewing of the external world without a decrease in light quantity. A half mirror may be disposed instead of the hologram optical element. In this case, however, the light quantity of the external world image is a half or less and the external world image as bright as the image obtained with the hologram optical element cannot be obtained.

In this embodiment, the hologram optical element 19 is attached to the plane but may alternatively be attached to an aspherical shape.

### INDUSTRIAL APPLICABILITY

The present invention is applicable to the display of an image to the wearer.

### REFERENCE SIGNS LIST

1 Head mounted display
2 Image display member
3 Image generation unit
4 Front photographing camera
5 First housing
6 Proximity sensor
10 Image display unit
20 Frame
21L Left temple part
21R Right temple part
22L, 22R Nose pad
24 Front part
24L Left front part
24R Right front part
25L Left corner connector
25R Right corner connector
37 External cover
38 Intermediate cable
60 Relay unit
61 External connection cable
61a Connector
62 Attachment member
70 Earphone unit
71 Second housing
72 Attachment member
73 Attachment opening
81 Relay circuit board
100 Mobile computer

1.-13. (canceled)

14. A head mounted display comprising:
an image display unit in which an image display member to be disposed in front of a wearer's eye and an image generation unit for generating an image to be displayed on the image display member are fixed to a housing, the housing containing a base end part of the image display member and the image generation unit;
a frame to be mounted on a head of the wearer, including a right front part and a left front part to be disposed on a front side of the head of the wearer, a right temple part connected to a right end of the right front part, a left temple part connected to a left end of the left front part, and supporting the image display unit;
a relay unit including a relay circuit board on which a sensor for detecting a state of the head of the wearer is mounted, and supported by the right temple part or the left temple part;
an external connection cable connected to the circuit board;
an intermediate cable for connecting between the circuit board and the image display unit; and
a second housing incorporating an additional device and detachably attached to the right temple part or the left temple part with an attachment member formed of an elastic material,
wherein transmission paths for signals and power between the image display unit and the unit connected to the external connection cable are formed by the external connection cable, the relay circuit board, and the intermediate cable, and transmission paths for signals and power between the sensor and the unit connected to the external connection cable are formed by the external connection cable and the relay circuit board, and
wherein the image display member is disposed in front of a right eye of the wearer, the image display unit is disposed in the right front part, the relay unit is disposed in the right temple part, and the second housing is disposed in the left temple part; or the image display member is disposed in front of a left eye of the wearer, the image display unit is disposed in the left front part, the relay unit is disposed in the left temple part, and the second housing is disposed in the right temple part.

15. The head mounted display according to claim 14, wherein a plurality of kinds of sensors for detecting the state of the head of the wearer is mounted on the relay circuit board.

16. The head mounted display according to claim 14, wherein the image display unit includes a proximity sensor that detects an object in front of the sensor.

17. The head mounted display according to claim 14, wherein the attachment member formed of the elastic material protrudes to an inside of one of the right temple part and the left temple part that corresponds to the temple part on which the second housing is mounted.

18. The head mounted display according to claim 14, wherein the attachment member formed of the elastic material is fixed to the second housing.

19. The head mounted display according to claim 14, having an attachment structure where one of the right temple part and the left temple part corresponding to the temple part on which the second housing is mounted is provided with an attachment opening, and the attachment member formed of the elastic material is internally fitted to the attachment opening.

20. The head mounted display according to claim 19, wherein when the attachment member formed of the elastic material is internally fitted to the attachment opening, a retaining end part having larger width than an inner width of the attachment opening goes through the attachment opening and is disposed inside the temple part.

21. The head mounted display according to claim 14, wherein the additional device is a wireless device.

22. The head mounted display according to claim 14, wherein the additional device is an earphone.

23. A wearable computer comprising:
the head mounted display according to claim 14; and
a mobile computer connected to the external connection cable of the head mounted display.

24. The wearable computer according to claim 23, wherein the mobile computer includes a wireless communication device.

25. The wearable computer according to claim 23, wherein the frame supports the image display unit on one of the right front part and the left front part, and the relay unit is supported by one of the right temple part and the left temple part that is on the same side as a side where the image display unit is supported.

26. The head mounted display according to claim 15, wherein the image display unit includes a proximity sensor that detects an object in front of the sensor.

27. The head mounted display according to claim 15, wherein the attachment member formed of the elastic material protrudes to an inside of one of the right temple part and the left temple part that corresponds to the temple part on which the second housing is mounted.

28. The head mounted display according to claim 15, wherein the attachment member formed of the elastic material is fixed to the second housing.

29. The head mounted display according to claim 15, having an attachment structure where one of the right temple part and the left temple part corresponding to the temple part on which the second housing is mounted is provided with an attachment opening, and the attachment member formed of the elastic material is internally fitted to the attachment opening.

30. The head mounted display according to claim 15, wherein the additional device is a wireless device.

31. The head mounted display according to claim 15, wherein the additional device is an earphone.

32. A wearable computer comprising:
the head mounted display according to claim 15; and
a mobile computer connected to the external connection cable of the head mounted display.

33. The head mounted display according to claim 16 wherein the attachment member formed of the elastic material protrudes to an inside of one of the right temple part and the left temple part that corresponds to the temple part on which the second housing is mounted.
